(19) United States
(12) Patent Application Publication
(10) Pub. No.: US 2003/0162725 A1
(43) Pub. Date: Aug. 28, 2003

(54) Synergistic pharmaceutical combinations for treating obesity with EGCG

(76) Inventors: Donald K. Riker, Chattanooga, TN (US); Michael Y. Law, Chattanooga, TN (US)

Correspondence Address: Douglas T. Johnson, Miller & Martin LLP, 1000 Volunteer Building, 832 Georgia Avenue, Chattanooga, TN, 37402, US

(21) Appl. No.: 10/373,214

(22) Filed: Feb. 24, 2003

Related U.S. Application Data
(60) Provisional application No. 60/360,199, filed on Feb. 25, 2002.

(51) Int. Cl. A61K 38/04; A61K 31/7048; A61K 31/405; A61K 31/353

(52) U.S. Cl. 514/19; 514/27; 514/419; 514/456

(57) ABSTRACT

This invention relates to a novel pharmaceutical composition which contains 5-hydroxytryptophan (5-HTP) with or without Vitamin B6 (pyridoxal phosphate) as a cofactor in combination with epigallocatechin gallate (EGCG) and caffeine. This pharmaceutical composition may be used for the treatment of obesity or appetite suppression.

### RELATED APPLICATIONS

This application claims priority to provisional application Ser. No. 60/360,199 filed Feb. 25, 2002.

### BACKGROUND OF THE INVENTION

1. Field of the Invention

This invention relates to a novel pharmaceutical composition which contains 5-hydroxytryptophan (5-HTP) with or without Vitamin B6 (pyridoxal phosphate) as a cofactor in combination with epigallocatechin gallate (EGCG) and caffeine and a method for the treatment of obesity and appetite suppression.

2. Description of Related Art

Studies have documented that medications which increase brain serotonin (5-hydroxytryptamine, 5-HT), such as fenfluramine, are effective anorectic agents to help obese patients lose weight and to decrease cravings for sweets and carbohydrates. 5-Hydroxytryptophan, abbreviated 5-HTP, is the immediate precursor of 5-HT. A goal of treatment with 5-HTP is to increase brain 5-HT. Previous studies in animals and humans have established that oral administration of 5-HTP increases brain 5-HT. It is desirable to have such an anorectic agent for human therapy which does not present the well-known unwanted and often dangerous effects typical of amphetamine and fenfluramine or their congeners, ranging from nausea and insomnia to hypertension and cardiac arrhythmias.

Medications which increase brain serotonin (5-HT) act to decrease hunger and cravings for food, especially for carbohydrates and sweets. The biosynthesis of 5-HT in the brain proceeds from the uptake of dietary tryptophan by the neuron, followed by its conversion to 5-hydroxytryptophan (5-HTP) by the enzyme tryptophan hydroxlyase. The main metabolic pathway to 5-HT is via decarboxylation of 5-HTP by the enzyme L-aromatic amino acid decarboxylase. Like other amino acid decarboxylases, this enzyme needs Vitamin B6 (pyridoxal phosphate) as a cofactor.

5-HTP is the direct precursor of serotonin. 5-HTP is a natural compound isolated from the seeds of an African plant called Griffonia simplicifolia, grown mostly in Ghana and the Ivory Coast. It can also be made synthetically in the laboratory. Its toxicity is extremely low as noted by lethal dose (LD) studies. Studies conducted in the rat and mouse have demonstrated that the LD.sub.50 is negligible as compared to therapeutic doses: LD50 per os in the mouse 2500 mg/kg; LD50 i.p. 1400 mg/kg. However, synthetically produced 5-HTP has been associated with a cluster of symptoms called eosinophilia-myalgia syndrome (EMS). EMS is a serious systemic illness characterized by elevations of certain white blood cells and severe muscle pain. In 1989 there was an epidemic outbreak of EMS, triggered by the consumption of synthetically produced L-tryptophan produced by a fermentation process. More than 1,500 cases including at least 37 deaths were reported to the CDC as of February, 2001. 5-HTP isolated from the seeds of Griffonia simplicifolia does not require the use of a fermentation process and is a safer product.

Green tea has long been known to have an effect on body weight and energy expenditure. Epigallocatechin gallate (abbreviated herein as EGCG) is a naturally-occurring substance found chiefly in green tea and green tea extracts. EGCG is ideal as a weight loss agent because of its lack of toxicity or apparent side effects. EGCG and related catechins occur naturally in several types of plants, including tea, and thus have a long history of safety in that form. Recently, the anorectic effects of green tea have been attributed to polyphenols, especially the most abundant one—EGCG (Dulloo A G, et al. Am J Clin Nutr 1999; 70:1040-5). The thermogenesis effect of EGCG has also been shown to be dependent on the sympathetically released norepinephrine (NE) in the activation of peripheral thermogenesis (Dulloo A G. et al. Int J Obes Relat Metab Disord 2000; 24:252-8). These studies indicate that it is not EGCG alone, but the combination of EGCG and caffeine via their interaction with sympathetically released NE that confers green tea with its ability to enhance thermogenesis. The mechanisms behind these synergistic interactions are to be expected because EGCG and caffeine act in concert along different control points underlying NE-induced thermogenesis. EGCG inhibits the enzyme catechol-O-methyltransferase that degrades NE within the synaptic cleft (Borehardt R T, Huber, J A, J Med Chem 1975; 18:120-2), whereas caffeine inhibits primarily the phosphodiesterase enzyme complex that degrades cyclic AMP, the intracellular secondary messenger for NE-mediated thermogenesis. The result is that two feedback inhibition pathways along the pathway of NE-activated thermogenesis have been removed. One would therefore expect the combination of EGCG and caffeine to be more effective than either compound alone in potentiating thermogenesis under sympathetic neural control.

In addition, there is evidence from studies that consumption of green tea (Camellia sinensis) will lower blood lipids levels, and it is known that high levels of circulating free fatty acids are often related to insulin resistance and thereby to erratic blood glucose control (Mitscher L A, et al. Med Res Rev 1997; 17:327-65). However, this is an indirect effect rather than a direct one; it is analogous to maintaining—accurately—that substantial weight loss improves insulin resistance in many individuals.

### SUMMARY OF THE INVENTION

The present invention recognizes and addresses the foregoing disadvantages, and others, of prior art compositions and methods.

Briefly, the present invention is a novel pharmaceutical composition for the treatment of obesity and appetite suppression. The applicants have discovered that administering 5-hydroxytryptophan (5-HTP) in combination with epigallocatechin gallate (EGCG) and caffeine to a patient results in unexpected potent anorectic activity that far exceeds the expected anorectic activity of combining 5-HTP and EGCG/caffeine. Additionally, the present invention provides an effective method for treating obesity and suppressing the appetite using naturally substances with little or no side effects.

One novel aspect of the invention is the use of 5-HTP and EGCG/caffeine to independently effect brain HT concentration and to control NE-activated thermogenesis.

A second novel aspect of the invention relates to the synergistic effect of using 5-HTP, EGCG, and caffeine to control weight loss. In particular, capsules containing 5-HTP (25 mg) plus EGCG (90 mg) and caffeine (50 mg) orally administered one to three times per day are particularly effective.

Accordingly, it is an object of the present invention to provide an appetite suppressant medication and method of use which provides unexpected and surprisingly potent anorectic activity.

More particularly, it is an object of the present invention to provide appetite suppressant medications having little or no adverse side effects.

More particularly, it is an object of the present invention to provide appetite suppressant medications which consist of naturally occurring substances, naturally derived 5-HTP and EGCG/caffeine.

Moreover, it is an object of the present invention to provide a method for the treatment of obesity and appetite suppression.

Additional objects and advantages of the invention will be set forth in part in the following description, or may be obvious from the description, or may be learned through practice of the invention.

### DETAILED DESCRIPTION OF THE INVENTION

The pharmaceutical compounds according to the present invention are found in two different classes of therapeutically active substances, i.e. one class comprising the precursor of the neurotransmitter serotonin, such as commercially available 5-hydroxytryptophan and its salts. It should be understood that any of its L, D or racemic forms are suitable, however, precursors are preferably in L form. The second class comprises EGCG or derivatives thereof (wherein “derivatives thereof” also encompasses natural products and their extracts containing same) that serve to promote NE-activated thermogenesis. Preferably, EGCG is used in concert with caffeine in a preferred ratio of 9:5 weight percent EGCG to caffeine. It should be understood that whenever in the present specification reference is made for the sake of simplicity to “EGCG”, the naturally occurring compound, i.e (−) epigallocatechin gallate, is meant. EGCG and derivatives thereof may occur as extracts of natural products containing EGCG at high concentrations, such as the extract of the leaves of Camellia sinensis or other extracts of natural products containing it.

The term “therapeutically active substance” as used herein is intended to mean any physiologically or pharmacologically active substance that produces a localized or systemic effect in animals, in particular in mammals, including humans, primates and domestic animals.

It has been found that it is preferable to administer to an individual in need thereof in a single or multiple regimen dose of at least 0.5 mg/day of 5-hydroxytryptophan and or an equivalent amount of a pharmacologically acceptable salt thereof. EGCG is available in purified form from various commercial suppliers. The compound can also be administered as an extract of green tea. Exact dosing will depend upon the form of EGCG used, the weight of the individual involved, and the other components of the diet.

This invention provides a pharmaceutical composition providing between about 0.5 mg to 5 g of 5-HTP, between about 0.5 mg to 6 g of EGCG, between about 0.5 mg to 6 g of caffeine, and between about 0.05 mg to 2 g of Vitamin B6 for administration up to 3 times daily. Because Vitamin B6 doses above 2 g per day may be toxic, high doses of Vitamin B6 are not recommended.

The preferred embodiment is a pharmaceutical tablet containing green tea leaf standardized extract consisting of 180 mg of antioxidant polyphenols, 90 mg of EGCG and 50 mg of naturally occurring caffeine, 25 mg of naturally derived 5-HTP and 2 mg of Vitamin B6 as pyridoxine hydrochloride. The tablet may be taken up to 3 times daily.

Additional ingredients may be added to the pharmaceutical composition, including, but not limited to vitamins, minerals and other trace elements. These supplements can be varied as desired but are typically equal to the RDA or greater based on 2,000 calories. A variety of herbal supplements may also be added to the pharmaceutical composition of the present invention including ginseng root and others.

The only reported side effect of this medication is nausea which is mild in severity. However, it has been found that the nausea is temporary and usually disappears during the second week of treatment.

The composition according to the present invention can be formulated for administration by any suitable route such as the oral, rectal, nasal, or parenteral administration route. Thus, the composition may be in the form of tablets, capsules, suspensions, emulsions, solutions, injectables, suppositories, sprays, aerosols or another suitable form.

Formulations for oral use include tablets, which contain the active ingredients in admixture with non-toxic pharmaceutically acceptable excipients. These excipients may be, for example, inert diluents, such as calcium carbonate, sodium chloride, lactose, calcium phosphate or sodium phosphate; granulating and disintegrating agents, for example, potato starch or alginic acid; binding agents, for example, starch, gelatin or acacia; and lubricating agents, for example, magnesium stearate, stearic acid or talc. Other pharmaceutically acceptable excipients can be colorants, flavouring agents, plasticizers, humectants etc. The tablets may be uncoated or they may be coated by known techniques, optionally to delay disintegration and absorption in the gastrointestinal tract and thereby provide a sustained action over a longer period. For example, a time delay material such as glyceryl monostearate or glyceryl distearate may be employed. Formulations for oral use may also be presented as chewing tablets, or as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil. Powders, dispersible powders or granules suitable for preparation of an aqueous suspension by addition of water are also convenient dosage forms of the present invention.

Formulations for oral use also include buccal/oral strips, which contain the active ingredients in portable, rapidly-dissolving complex carbohydrate or starch-based strips. These strips consist of active ingredients, flavorings, colors or excipients that rapidly dissolve in the buccal-oral cavity. These strips are portable and small and do not require swallowing a pill or liquid. Alternatively, the strips may be dissolved directly into food or drinks.

Formulation as a suspension provides the active ingredients in admixture with a dispersing or wetting agent, suspending agent and one or more preservatives. Suitable dispersing or wetting agents are, for example, naturally-occurring phosphatides, as e.g. lecithin, or condensation products of ethylene oxide with e.g. a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc. Suitable suspending agents are, for example, sodium carboxymethylcellulose, methylcellulose, sodium alginate etc. The pharmaceutical formulation may also be administered parenterally (intravenous, intramuscular, subcutaneous or the like) in dosage forms or formulations containing conventional, non-toxic pharmaceutically acceptable carriers and adjuvants.

Preferably, the pharmaceutical composition of the present invention comprises a combination product containing naturally derived 5-hydroxytryptophan with or without Vitamin B6 (pyridoxal phosphate) as a cofactor in combination with EGCG and caffeine, i.e. in the case of a tablet; one tablet comprises a mixture of the four active components. However, the pharmaceutical composition of the present invention may also be presented in one package comprising two separate containers, one container comprising dosage form of the 5-hydroxytryptophan/Vitamin B6 and the other container comprising a dosage form of the EGCG/caffeine. In such cases, special instructions for substantially concomitant use of the two drugs should be enclosed with the product. The two dosage forms can be the same or they may be different, preferably the two dosage forms are the same.

It is to be understood that according to the teachings of the invention, the invention can be practiced by administering serotonin-mediated neurotransmission stimulating compounds, for example, 5-hydroxytryptophan, to a subject as a single dose one or more times per day, or as a plurality of unit doses one or more times per day without deviating from the teachings of the invention.

It is to be further understood that the present invention also includes a method of making a pharmaceutical composition for the treatment of obesity or appetite suppression, wherein the method comprises a step of mixing a serotonin precursor such as 5-hydroxytryptophan with EGCG.

In one aspect the present invention relates to a method for treatment of overweight or obese individuals, in particular in humans, or for reducing the adipose tissue mass/lean mass body mass ratio of an individual, in particular a human.

In the present context the term “overweight” is used as an indication of a body with a weight exceeding the “desirable weight”, whereas the term “obesity” is used when the body weight is 20% or more above the “desirable weight.” Desirable weights for humans are defined as a body mass index less than or equal to 24.

In another aspect, the invention can be used along with other appetite suppressant drugs, such as N,N-dimethyl-1-[1-(4-chlorophenyl)cyclobutyl]-3-methylbutylamine hydrochloride monohydrate (sibutramine hydrochloride), synephrine, amphetamine, phentermine, diethylpropion, phendimetrazine, ephedrine or similarly-acting agents to increase the weight loss which would occur with the use of these agents alone.

In addition, the invention can be used with other drugs that effect fat uptake to enhance the effect of the invention, for example, lipase inhibiting drugs such as Orlistat, and Xenical, or lipid absorbing polysaccharides such as Chitosan, or alpha amylase inhibitors such as acarbose, voglibose, miglitol, emiglitate, camiglibose, salbostatin that reduce starch intake in humans. However, the pharmaceutical composition of the present invention does not require the use of drugs that effect fat uptake or active ingredients such as phenylpropanolamine, ephedrine, ephedra alkaloids, ma huang (Chinese ephedra) or citrus aurantium (bitter orange peel).

In a further aspect, the present invention also relates to the use of a combination of 5-hydroxytryptophan, Vitamin B6 (pyridoxal phosphate) as a cofactor, EGCG and caffeine for the manufacture of a pharmaceutical composition for appetite suppression, the treatment of obesity or diseases aggravated thereof.

The invention is further illustrated by means of the following illustrative embodiment, which is given for the purpose of illustration only and is not meant to limit the invention to the particular components and amounts disclosed. The following example shows the preferred embodiment for producing appetite suppressant medications comprising 5-hydroxytryptophan with or without Vitamin B6 (pyridoxal phosphate) as a cofactor in combination with EGCG and caffeine.

One coated tablet of the preferred embodiment of the pharmaceutical composition according to the present invention has the following composition.

| Ingredient | Mg/caplet | % by Weight |
|---|---|---|
| Active Ingredients: | | |
| Vitamin B6 (pyridoxine hydrochloride) | 26.000 | 2.680% |
| Green tea leaf extract | 600.000 | 61.856% |
| Marker: Caffeine | (50.000) | |
| Marker: Epigallocatechin gallate | (90.000) | |
| (EGCG) | | |
| Panax ginseng root (4% ginsenosides) | 5.000 | 0.515% |
| from 80% Marker: Ginsenosides | | |
| | (4.000) | |
| 5-Hydroxytryptophan (from 95%) | 26.500 | 2.732% |
| Inactive Ingredients: | | |
| Dicalcium phosphate | 70.500 | 7.268% |
| Microcrystalline cellulose | 170.000 | 17.526% |
| Croscarmellose sodium | 36.000 | 3.711% |
| Stearic acid | 24.000 | 2.474% |
| Silicon dioxide | 6.000 | 0.619% |
| Magnesium stearate | 6.000 | 0.619% |
| Total Weight of core tablet: | 970.000 | 100.000% |
| Dri-Klear Clear 010 (53-859010-00) | 13.000 | |
| HPMC Powder | | |
| Total Weight of coated tablet: | 983.000 | |

We claim:

1. A pharmaceutical composition comprising an effective amount of:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound that promotes thermogenesis, wherein said compound is selected from the group consisting of epigallocatechin gallate or derivatives thereof.

2. The pharmaceutical composition according to claim 1, wherein the compound which enhances serotonin-mediated neurotransmission, or a pharmaceutically acceptable salt thereof is 5-hydroxytryptophan.

3. The pharmaceutical composition according to claim 1, wherein the compound which enhances serotonin-mediated neurotransmission, or a pharmaceutically acceptable salt thereof is naturally derived 5-hydroxytryptophan.

4. The pharmaceutical composition according to claim 1, wherein the compound which enhances serotonin-mediated neurotransmission, or a pharmaceutically acceptable salt thereof is selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxy-L-tryptophyl-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alaninc hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and combinations thereof.

5. The pharmaceutical composition according to claim 1, comprising between about 0.5 mg to about 5 g of 5-hydroxytryptophan.

6. The pharmaceutical composition according to claim 1, comprising between about 0.5% to about 15% by weight 5-hydroxytryptophan.

7. The pharmaceutical composition according to claim 1, comprising between about 0.5 mg to 6 g of epigallocatechin gallate.

8. The pharmaceutical composition according to claim 1, comprising between about 0.5% to about 25% by weight epigallocatechin gallate.

9. The pharmaceutical composition according to claim 1, further comprising an effective amount of caffeine.

10. The pharmaceutical composition according to claim 9, comprising between about 0.5 mg to 6 g caffeine.

11. The pharmaceutical composition according to claim 1, further comprising an effective amount of Vitamin B6.

12. The pharmaceutical composition according to claim 11, comprising between about 0.05 mg to 2 g of Vitamin B6.

13. The pharmaceutical composition according to claim 1, further comprising an effective amount of other anorectic agents selected from the group consisting of phentermine, diethylpropion, phendimetrazine and ephedrine.

14. A pharmaceutical composition comprising:
(a) At least 0.5 mg of 5-hydroxytryptophan, and
(b) At least 0.5 mg of epigallocatechin gallate or any derivative thereof.

15. A method for the treatment of obesity or inducing weight loss in human subjects comprising administration of a pharmaceutical composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound that promotes thermogenesis, wherein said compound is selected from the group consisting of epigallocatechin gallate or derivatives thereof.

16. The method according to claim 15, wherein the compound which enhances serotonin-mediated neurotransmission, or a pharmaceutically acceptable salt thereof is 5-hydroxytryptophan.

17. The method according to claim 15, wherein the compound which enhances serotonin-mediated neurotransmission, or a pharmaceutically acceptable salt thereof is naturally derived 5-hydroxytryptophan.

18. The method according to claim 15, wherein compound which enhances serotonin-mediated neurotransmission, or a pharmaceutically acceptable salt thereof is selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxyl-L-tryptophyl-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alaninc hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and combinations thereof.

19. The pharmaceutical composition according to claim 15, comprising between about 0.5 mg to about 5 g of 5-hydroxytryptophan.

20. The pharmaceutical composition according to claim 15, comprising between about 0.5% to about 15% by weight 5-hyydroxytryptophan.

21. The method according to claim 15, comprising between about 0.5 mg to 6 g of epigallocatechin gallate.

22. The method according to claim 15, comprising between about 0.5% to about 25% by weight epigallocatechin gallate.

23. The method according to claim 15, wherein the pharmaceutical composition further comprises an effective amount of caffeine.

24. The method according to claim 15, wherein the pharmaceutical composition further comprises an effective amount of Vitamin B6.

25. The method according to claim 15, further comprising administration of an effective amount of other anorectic agents selected from the group consisting of phentermine diethylpropion, phendimetrazine and ephedrine.

26. The method according to claim 15, wherein the compound which enhances serotonin-mediated neurotransmission, or a pharmaceutically acceptable salt thereof is administered at a dose ranging from about 0.5 mg/day to about 5 g/day and the compound that promotes thermogenesis is administered at a dose ranging from 0.5 mg to 6 g per day.

27. A method for suppressing the appetite of human subjects comprising administration of a pharmaceutical composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound that promotes thermogenesis, wherein said compound is selected from the group consisting of epigallocatechin gallate or derivatives thereof.

28. The method according to claim 27, wherein the compound which enhances serotonin-mediated neurotransmission, or a pharmaceutically acceptable salt thereof is 5-hydroxytryptophan.

29. The method according to claim 27, wherein the compound which enhances serotonin-mediated neurotransmission, or a pharmaceutically acceptable salt thereof is naturally derived 5-hydroxytryptophan.

30. The method according to claim 27, wherein compound which enhances serotonin-mediated neurotransmission, or a pharmaceutically acceptable salt thereof is selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxyl-L-tryptophyl-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alaninc hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and combinations thereof.

31. The pharmaceutical composition according to claim 27, comprising between about 0.5 mg to about 5 g of 5-hydroxytryptophan.

32. The pharmaceutical composition according to claim 27, comprising between about 0.5% to about 15% by weight 5-hyydroxytryptophan.

33. The method according to claim 27, comprising between about 0.5 mg to 6 g of epigallocatechin gallate.

34. The method according to claim 27, comprising between about 0.5% to about 25% by weight epigallocatechin gallate.

35. The method according to claim 27, wherein the pharmaceutical composition further comprises an effective amount of caffeine.

36. The method according to claim 27, wherein the pharmaceutical composition further comprises an effective amount of Vitamin B6.

37. The method according to claim 27, further comprising administration of an effective amount of other anorectic agents selected from the group consisting of phentermine diethylpropion, phendimetrazine and ephedrine.

38. The method according to claim 27, wherein the compound which enhances serotonin-mediated neurotransmission, or a pharmaceutically acceptable salt thereof is administered at a dose ranging from about 0.5 mg/day to about 5 g/day and the compound that promotes thermogenesis is administered at a dose ranging from 0.5 mg to 6 g per day.

39. A method for the reduction of body mass of human subjects comprising administration of a pharmaceutical composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound that promotes thermogenesis, wherein said compound is selected from the group consisting of epigallocatechin gallate or derivatives thereof.

40. A method for the treatment of stress comprising administration of a pharmaceutical composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound that promotes thermogenesis, wherein said compound is selected from the group consisting of epigallocatechin gallate or derivatives thereof.

41. A pharmaceutical composition comprising:
(c) between about 0.5% to 15% by weight of a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(d) between about 0.5% to 25% by weight of a compound that promotes thermogenesis, wherein said compound is selected from the group consisting of epigallocatechin gallate or derivatives thereof.
